(19) United States
(12) Patent Application Publication
(10) Pub. No.: US 2024/0312200 A1
(43) Pub. Date: Sep. 19, 2024

(54) MEDICAL IMAGE PROCESSING APPARATUS AND METHOD FOR OPERATING THE SAME

(71) Applicant: FUJIFILM Corporation, Tokyo (JP)

(72) Inventor: Misaki GOTO, Kanagawa (JP)

(73) Assignee: FUJIFILM Corporation, Tokyo (JP)

(21) Appl. No.: 18/675,095

(22) Filed: May 27, 2024

Related U.S. Application Data
(63) Continuation of application No. PCT/JP2022/037653, filed on Oct. 7, 2022.

(30) Foreign Application Priority Data
Nov. 30, 2021 (JP) 2021-194738

(51) Int. Cl. G06V 10/98; G06T 7/00

(52) U.S. Cl. CPC G06V 10/987; G06T 7/0012; G06T 2207/30096

(57) ABSTRACT

Recognition processing of a specific photographic subject is executed on a medical image, and an acceptance mode is determined on the basis of a calculated reliability level of the recognition result of the specific photographic subject. The acceptance mode accepts a correction to the recognition result in accordance with the reliability level with respect to a threshold value, and the recognition result that is corrected is displayed. The acceptance mode includes a first mode for selecting an option of a candidate correction, a second mode for accepting no correction, and a third mode for accepting a correction using any text.

### CROSS-REFERENCE TO RELATED APPLICATIONS

This application is a Continuation of PCT International Application No. PCT/JP2022/037653 filed on 7 Oct. 2022, which claims priority under 35 U.S.C § 119(a) to Japanese Patent Application No. 2021-194738 filed on 30 Nov. 2021. The above application is hereby expressly incorporated by reference, in its entirety, into the present application.

### BACKGROUND OF THE INVENTION

### 1. Field of the Invention

The present invention relates to a medical image processing apparatus and a method for operating the same.

### 2. Description of the Related Art

When a medical imaging device such as an endoscope performs treatment tool recognition, lesion detection, or the like by using inference based on machine learning, consecutive recognition errors may occur for a patient or case for which such operations are difficult to perform. If consecutive recognition errors occur, incorrect recognition results are continuously displayed and recorded, which may confuse a user or increase the burden on the user due to an increase in the labor of correcting the recorded results later. For this reason, it is necessary to detect a possibility of erroneous recognition when a medical image is acquired.

Specifically, in JP6246431B (corresponding to US2018/249900A1), a detected candidate lesion region is subjected to emphasis processing to generate an image that allows a surgeon to estimate the probability of erroneous detection of the candidate lesion region. In the emphasis processing, a candidate lesion region detected from a predetermined feature value is colored in accordance with likelihood information (parameter), and when the detection of the same candidate lesion region continues continuously or intermittently for a certain period of time, a notification image is displayed by highlighting using the likelihood information to notify the surgeon that the candidate lesion region is present. In JP2021-115238A, text/image data is acquired from a paper medium on which ocular examination results of a subject are printed, and a user corrects any erroneous recognition result included in the acquired results, as appropriate. In image recognition, matching processing using erroneous recognition results accumulated by machine learning is performed, and a result with a low degree of matching is determined to be erroneous recognition.

### SUMMARY OF THE INVENTION

In JP6246431B, the number of consecutive recognitions of a detected candidate lesion region is used to change the display style of an image, and an image is displayed from which the probability of erroneous detection can be estimated. In JP2021-115238A, erroneous recognition is determined by matching processing using machine learning, and a user corrects an erroneous recognition result on a confirmation correction screen. In JP6246431B and JP2021-115238A, the possibility of erroneous detection or erroneous recognition of an image is displayed. JP6246431B provides no description concerning correction for the erroneous detection. JP2021-115238A allows acceptance of a correction to an acquired incorrect result from the user; however, it is inefficient for the user to perform correction by inputting any text each time the correction is performed. For this reason, it is desirable to efficiently correct a recognition result of a medical image acquired by examination using an endoscope, ultrasound, or the like, with a low burden on the user.

An object of the present invention is to provide a medical image processing apparatus that calculates a reliability level indicating a possibility of erroneous recognition of a recognition result of a specific photographic subject in a medical image, reduces a burden on a user on the basis of the reliability level, and efficiently corrects the recognition result, and a method for operating the medical image processing apparatus.

A medical image processing apparatus of the present invention includes a processor configured to acquire at least one medical image; execute recognition processing on the medical image to recognize a specific photographic subject; calculate a reliability level of a recognition result of the specific photographic subject; determine, based on the reliability level, an acceptance mode for accepting a correction to the recognition result; accept the correction to the recognition result in the determined acceptance mode; and display the recognition result that is corrected.

Preferably, the acceptance mode includes a first mode for accepting a correction to the recognition result from a user and automatically confirming the correction to the recognition result when a predetermined condition is satisfied. Preferably, the first mode displays, as options, candidate corrections to the recognition result, and allows the user to select a correction to the recognition result from the candidate corrections.

Preferably, the acceptance mode includes a second mode for confirming the recognition result without accepting a correction to the recognition result from the user. Preferably, the acceptance mode is determined to be the first mode when the reliability level is less than a first threshold value, and the acceptance mode is determined to be the second mode when the reliability level is greater than or equal to the first threshold value.

Preferably, the acceptance mode includes a third mode for accepting a correction to the recognition result from the user and manually confirming the recognition result. Preferably, the acceptance mode is determined to be the first mode when the reliability level is greater than or equal to a second threshold value, and the acceptance mode is determined to be the third mode when the reliability level is less than the second threshold value.

Preferably, time-series images that are the medical images in time series are acquired, the recognition processing is performed for each of the medical images constituting the time-series images, and the reliability level is calculated based on a number of recognitions of the specific photographic subject with respect to a number of the medical images constituting the time-series images. Preferably, the reliability level is calculated based on a number of recognitions of a photographic subject different from the specific photographic subject with respect to the number of medical images constituting the time-series images.

Preferably, in the recognition processing, first recognition processing for recognizing the specific photographic subject and second recognition processing for recognizing a photographic subject different from the specific photographic subject are executed, a first recognition result is acquired by the first recognition processing, a second recognition result is acquired by the second recognition processing, a correspondence between the first recognition result and the second recognition result is checked, and a first reliability level of the first recognition result and a second reliability level of the second recognition result are calculated.

Preferably, the recognition processing is executed on the medical image for the plurality of mutually different specific photographic subjects, recognition results including the recognition result are acquired, and the reliability level is calculated for each of the recognition results.

Preferably, at least one of a first threshold value of the reliability level or a second threshold value lower than the first threshold value is set for each type of the specific photographic subject, and the acceptance mode is determined based on the reliability level for at least one of the first threshold value or the second threshold value.

Preferably, acceptance of the correction and a display style of a screen are controlled in a stepwise manner, based on the reliability level.

Preferably, a button operation, a foot pedal operation, and a voice input are accepted for the correction.

A method for operating a medical image processing apparatus of the present invention includes a step of acquiring at least one medical image, a step of executing recognition processing on the medical image to recognize a specific photographic subject, a step of calculating a reliability level of a recognition result of the specific photographic subject, a step of determining, based on the reliability level, an acceptance mode for accepting a correction to the recognition result, a step of accepting the correction to the recognition result in the determined acceptance mode, and a step of displaying the recognition result that is corrected.

According to the present invention, it is possible to calculate a reliability level indicating a possibility of erroneous recognition of a recognition result of a specific photographic subject in a medical image, reduce a burden on a user on the basis of the reliability level, and efficiently correct the recognition result.

### BRIEF DESCRIPTION OF THE DRAWINGS

FIG. 1 is an explanatory diagram illustrating devices connected to a medical image processing apparatus;

FIG. 2 is a block diagram illustrating functions of the medical image processing apparatus;

FIG. 3 is an explanatory diagram of a medical image acquired in recognition processing;

FIG. 4 is an explanatory diagram of a medical image group on which the recognition processing is to be performed;

FIG. 5 is an explanatory diagram of the recognition processing for a medical image group;

FIG. 6 is an explanatory diagram of a first mode serving as an acceptance mode;

FIG. 7 is an explanatory diagram of a second mode serving as an acceptance mode;

FIG. 8 is an explanatory diagram of a third mode serving as an acceptance mode;

FIG. 9 is an explanatory diagram of features for the respective acceptance modes;

FIGS. 10A, 10B, and 10C are explanatory diagrams of a modification of acceptance mode control;

FIG. 11 is a flowchart illustrating a series of operations according to the present invention;

FIG. 12 is an explanatory diagram of a function of determining a reliability level, which is implemented in a second embodiment;

FIG. 13 is an explanatory diagram illustrating control of an acceptance mode by using recognition results of time-series images in the second embodiment;

FIG. 14 is an explanatory diagram of the functions of a recognition processing unit and a reliability level determination unit implemented in a third embodiment;

FIG. 15 is an explanatory diagram of the correspondence between a detected recognition result and a scene determination result in the third embodiment; and

FIG. 16 is an explanatory diagram of a screen for accepting a correction to a mismatch correspondence in the third embodiment.

### DESCRIPTION OF THE PREFERRED EMBODIMENTS

### First Embodiment

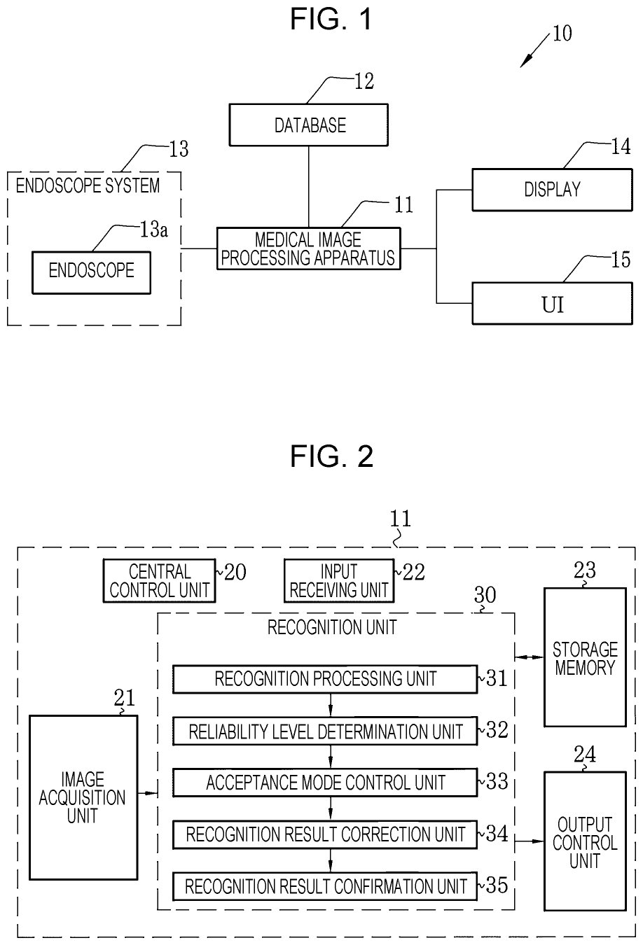

FIG. 1 is a diagram illustrating devices connected to a medical image processing apparatus 11 in a medical image processing system 10 according to an embodiment of the present invention. The medical image processing system 10 has the medical image processing apparatus 11, a database 12, an endoscope system 13 including an endoscope 13a, a display 14, and a user interface (UI) 15. The medical image processing apparatus 11 is electrically connected to the database 12, the endoscope system 13, the display 14, and the user interface 15.

The database 12 is a device capable of storing acquired images and transmitting and receiving data to and from the medical image processing apparatus 11, and may be a recording medium such as a USB (Universal Serial Bus) or an HDD (Hard Disc Drive). The display 14 displays an image acquired by the medical image processing apparatus 11. The user interface 15 is an input device for performing input and the like to the medical image processing apparatus 11, and may be implemented using a foot pedal, a gesture recognizer, or a voice recognizer in addition to or instead of a keyboard and a mouse. The input may be performed using not only the user interface 15 but also an input means included in a medical device, such as a switch of the endoscope 13a.

The database 12 stores moving images and still images for medical examination, which are generated by the endoscope system 13 or other medical devices. In imaging for medical examination, white illumination light is used, a video signal of 60 frames per second (60 fps) is acquired, and the imaging time is recorded, unless otherwise specified. In the case of a video signal of 60 fps, it is preferable to count the time in increments of 1/100 seconds.

As illustrated in FIG. 2, in the medical image processing apparatus 11, a central control unit 20, which is constituted by an image control processor, operates a program in a program memory to implement the functions of an image acquisition unit 21, an input receiving unit 22, a storage memory 23, an output control unit 24, and a recognition unit 30. With the implementation of the functions of the recognition unit 30, the functions of a recognition processing unit 31, a reliability level determination unit 32, an acceptance mode control unit 33, a recognition result correction unit 34, and a recognition result confirmation unit 35 are implemented. The image acquisition unit 21 receives data of an image acquired by the endoscope system 13, images stored in the database 12, and the like and transmits the data to the recognition unit 30.

The input receiving unit 22 is connected to the user interface 15. The storage memory 23 temporarily stores an image on which recognition processing is to be performed. The function of temporary storage may be included in the database 12 instead of the storage memory 23. The output control unit 24 performs control to display an image on the display 14. In the medical image processing apparatus 11, a program related to processing such as image processing is stored in a program memory (not illustrated).

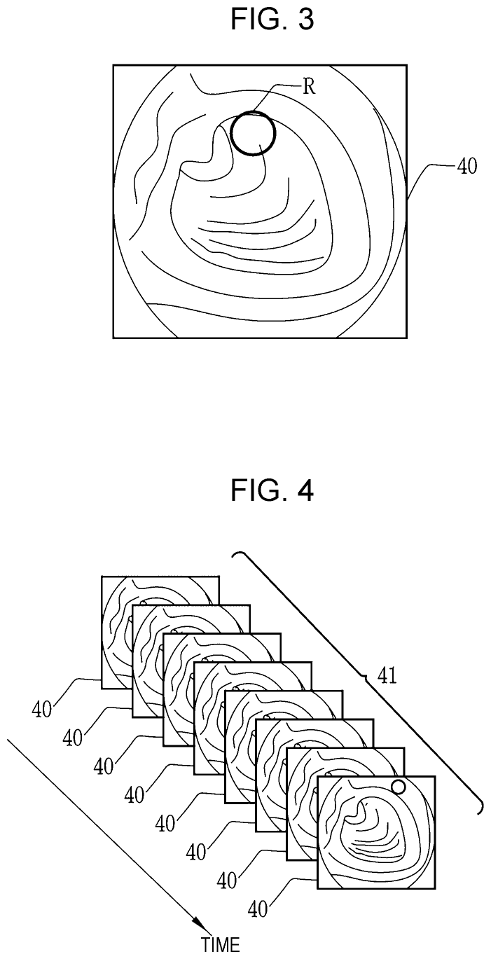

As illustrated in FIG. 3, in the recognition processing, a preset recognition result indicating the presence or absence and name of a specific photographic subject that a medical image 40 has is acquired. The medical image 40 is an image of the inside of a living body captured with the endoscope 13a or the like. The specific photographic subject is a target to be recognized in the recognition processing. Examples of the specific photographic subject include a type of lesion such as a tumor or inflammation detected as a region of interest R, a treatment tool S such as forceps or a snare, and an observed site or organ such as the pylorus of the stomach or the rectum. For the medical image 40 after the recognition processing, a correction to the recognition result is accepted on the basis of a reliability level described below. The medical image 40 is a frame constituting a moving image or is a still image.

As illustrated in FIG. 4, as data to be input to the recognition unit 30, one image may be input for each recognition processing, or time-series medical images 40 or a medical image group 41 obtained by grouping a plurality of medical images 40 may be input. The time-series medical images 40 refer to a plurality of temporally consecutive medical images 40. The medical image group 41 on which the recognition processing is to be performed is used in endoscopy such that the endoscopy is performed on the basis of not the entire video but preferably the video of divided ranges or narrowed ranges. To perform the recognition processing, the medical image processing apparatus 11 acquires the medical images 40 or the medical image group 41 from the image acquisition unit 21 and transmits the medical images 40 or the medical image group 41 to the recognition unit 30.

The recognition processing unit 31 executes recognition processing of the specific photographic subject in the medical images 40 or the medical image group 41 received by the recognition unit 30 by using content learned in advance. The recognition processing unit 31 has a function of a trained model necessary for the recognition processing. That is, the recognition processing unit 31 is a computer algorithm constituted by a neural network that performs machine learning, and determines the presence or absence of the specific photographic subject for each of the input medical images 40 in accordance with learning content, or specifically infers the specific photographic subject when each of the medical images 40 has the specific photographic subject, to acquire a recognition result. As the recognition result, in addition to the name of the specific photographic subject, information such as the rate of match with learning content learned in advance is also acquired. The rate of match is used to calculate a reliability level.

In the recognition processing, not a single result but a plurality of results can be recognized. For example, a recognition result of “tumor: 95%” or recognition results of “tumor: 65%; bleeding: 20%; and gastritis: 10%” may be obtained for the region of interest R.

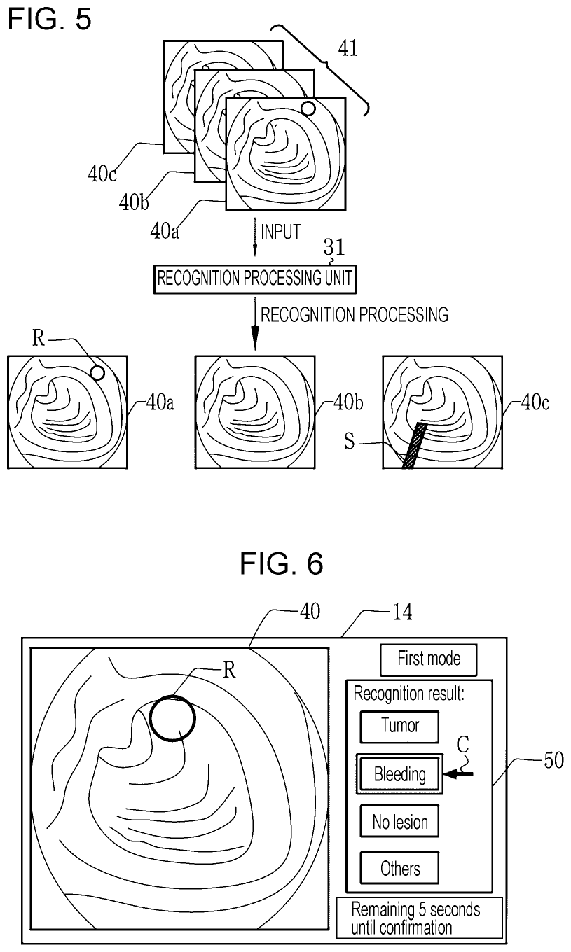

As illustrated in FIG. 5, when the medical image group 41 is input to the recognition processing unit 31, in the inference based on machine learning, the recognition processing unit 31 determines whether each of the medical images 40 constituting the medical image group 41 has the specific photographic subject, which is learned in advance. If each of the medical images 40 has the specific photographic subject, the recognition processing unit 31 performs recognition processing for the name of the learned specific photographic subject, and acquires a recognition result. Examples of the recognition result include recognition of the region of interest R such as a lesion in a medical image 40a, non-recognition of the specific photographic subject in a medical image 40b, and recognition of the treatment tool S such as a snare in a medical image 40c. The reliability of the recognition result is represented by a reliability level. The reliability level and the recognition result are associated with the medical image 40 and are transmitted to the reliability level determination unit 32. The medical image group 41, which is a frame image group or a still image group, may be input to the recognition processing unit 31, or the medical images 40 may be individually input one by one to the recognition processing unit 31.

The reliability level determination unit 32 determines the reliability level of the recognition result acquired by the recognition processing unit 31. The reliability level is determined using the rate of match with the learning content learned in advance, which is calculated by the recognition processing unit 31, the rate of erroneous detection for each recognition target, image quality information, and the like. A high reliability level indicates a low probability that the recognition result is correct, that is, a low probability that the recognition result needs to be corrected. A low reliability level indicates a high probability that the recognition result is incorrect, that is, a high probability that the recognition result needs to be corrected. The reliability level is represented by, for example, percentage (%).

The acceptance mode control unit 33 controls the display styles of the recognition results of the medical images 40 and the acceptance of a correction to the recognition results on the basis of the determined reliability levels. An acceptance mode is determined in accordance with the reliability level of the recognition result acquired for each medical image. The medical images 40 are displayed together with the recognition results in the display styles for the respective acceptance modes. The acceptance modes will be described below.

The recognition result correction unit 34 accepts a correction to the recognition results in accordance with the acceptance modes. The correction involves selecting an option from among a plurality of candidate recognition results that are detected or inputting the content of the correction in any text. The correction is performed by user input via the user interface 15, and the input content is reflected on the display 14. If no correction is accepted, the recognition result correction unit 34 is not used. The content of the correction is stored in response to the recognition result confirmation unit 35 accepting a confirmation instruction.

In accordance with an instruction to confirm a recognition result for each acceptance mode, the recognition result confirmation unit 35 confirms the recognition result. The confirmed results are stored in association with the medical images 40. The medical images 40 may be stored by transmitting the medical images 40 to the database 12 one by one, or may be temporarily stored in the storage memory 23 and then collectively transmitted to the database 12.

The output control unit 24 controls the display style of the display 14 in accordance with the acceptance modes determined by the acceptance mode control unit 33 on the basis of the reliability levels. It is preferable to, in response to an instruction to confirm the recognition result of the medical image 40 for each acceptance mode, end the acceptance mode and perform a display indicating that the medical image 40 is stored together with the recognition result. After the recognition result is confirmed and stored, the recognition result of the next medical image 40 is acquired, and the display is switched to the display of the acceptance mode for each reliability level.

The recognition processing for the medical images 40 will be described. The medical images 40 acquired by the image acquisition unit 21 from the database 12 or the endoscope system 13 are transmitted to the recognition unit 30, and are subjected to recognition processing. Through the recognition processing, recognition results of the specific photographic subject in the medical images 40 are acquired, and the reliability levels thereof are calculated. The medical images 40 subjected to the recognition processing are displayed on the display 14 in different acceptance modes in accordance with the calculated reliability levels.

The reliability levels are indices indicating the reliability of recognition results calculated by inference using a learning model, and are preferably represented by using percentage (%). The control of acceptance of corrections in accordance with the reliability levels is implemented by switching the acceptance mode. The reliability level for each of the medical images 40 to be used for switching the acceptance mode is the highest one of the reliability levels calculated for the respective items such as the lesion name acquired in the recognition processing. For example, when the recognition results of the medical image 40 subjected to the recognition processing are “tumor: 65%; bleeding: 20%; and gastritis: 15%”, the medical image 40 is treated as a recognition result having a reliability level of 65%. In this case, screen display and acceptance of a correction to the recognition result are performed in the acceptance mode corresponding to a reliability level of 65%. An item with a low reliability level, for example, a reliability level less than 10%, need not be added to the recognition results.

The determination of the acceptance mode in accordance with the reliability level will be described. The acceptance mode is determined on the basis of the reliability level of a recognition result for the specific photographic subject, which is acquired by the recognition processing for each of the medical images 40, and the medical image 40 is displayed on the display 14. A correction to the acquired recognition result is accepted by using a correction method controlled for each acceptance mode. The corrected recognition result is displayed on the display 14.

The acceptance mode is set in a stepwise manner on the basis of the reliability level of each of the medical images 40 after the recognition processing is performed. The style of the screen display differs for each acceptance mode, and is a display style corresponding to the content of the accepted correction for the acceptance mode. The reliability level indicates the probability of erroneous recognition, and a recognition result with a reliability level close to 100% has a low necessity of correction, whereas a recognition result with a reliability level lower than 50% has a possibility of erroneous recognition. The acceptance mode is controlled so as to restrict acceptance of a correction for the medical image 40 associated with a recognition result with a high reliability level and accept a correction for the medical image 40 associated with a recognition result with a low reliability level, and the acceptance state of a correction is reflected in the screen display for each acceptance mode. A display style for accepting a correction is determined on the basis of the reliability level.

The acceptance mode is determined in accordance with the reliability level of a recognition result detected by the recognition processing. Specifically, any three types of patterns, namely, a first mode for a reliability level having a value within a predetermined range, a second mode for a reliability level having a value larger than the predetermined range, and a third mode for a reliability level having a value smaller than the predetermined range, are determined. The predetermined range of the reliability level is, for example, 50% or more and less than 90%.

As illustrated in FIG. 6, when the reliability level of the medical image 40 calculated in the recognition processing falls within the predetermined range, the acceptance mode is switched to the first mode in which a user input to correct the recognition result is accepted and the correction to the recognition result is automatically confirmed if a predetermined condition is satisfied. The predetermined condition is, for example, the lapse of a certain period of time. The predetermined condition can be set in advance for each recognition result in accordance with the importance of the recognition result. For example, when the recognition result is “tumor”, the recognition result may be automatically confirmed after a lapse of 30 seconds, and when the recognition result is “inflammation”, the recognition result may be automatically confirmed after a lapse of 10 seconds.

In FIG. 6, in the first mode, the medical image 40 subjected to the recognition processing and items such as lesion names for the acquired specific photographic subject are displayed on the display 14 in a recognition result display field 50 in a first mode screen. A displayed item is selected by user input to determine a recognition result. For example, when the recognition results for the region of interest R are “tumor: 65%”, “bleeding: 20%”, and “no lesion: 10%”, options of three items, namely, tumor, bleeding, and no lesion, and “others” are displayed on the display 14, and one of the options is selected by user input. When the option “others” is selected, the recognition result may be corrected to “unknown”, or the acceptance mode may be switched to the third mode for accepting input of any text described below. The selection of an option by user input is performed using a method such as operating a cursor C being displayed on the display 14 with the mouse.

In the first mode, the recognition result is confirmed not when any of the options is selected but when the predetermined condition is satisfied. If none of the options is selected when the predetermined condition is satisfied, the correction candidate having the highest reliability level is automatically confirmed. For this reason, in a case where the predetermined condition is the lapse of a certain period of time, it is possible to reselect an option within the certain period of time, and it is possible to prevent too much time from being spent on determination because any one of the correction candidates is difficult to select. If the recognition result is confirmed after the lapse of a certain period of time, a countdown to the confirmation may be displayed on the display 14. When any one of the correction candidates is selected, the selected correction candidate is confirmed as the content of the correction after the certain period of time has elapsed since the selection.

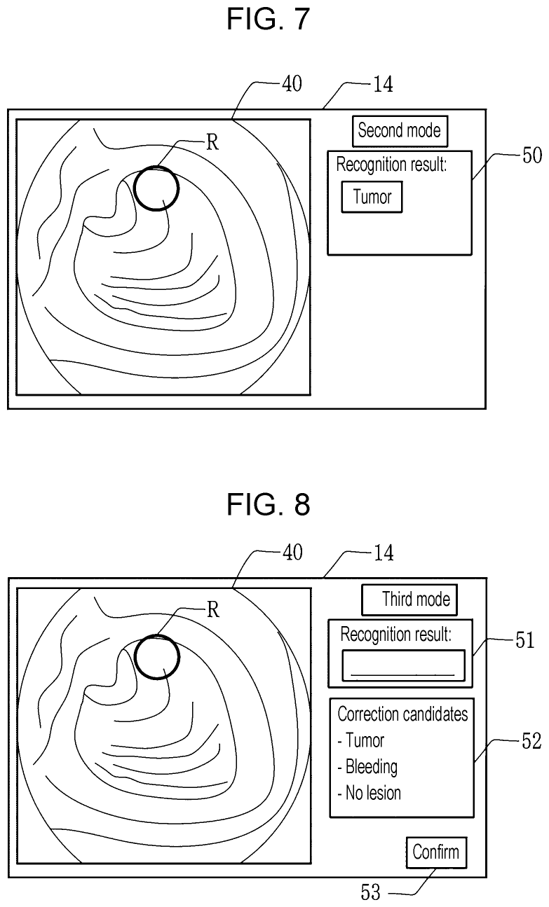

As illustrated in FIG. 7, when the reliability level has a value higher than the predetermined range, the acceptance mode is switched to the second mode in which the recognition result is confirmed without acceptance of a correction. The display 14 displays the medical image 40 subjected to the recognition processing and the confirmed recognition result in the recognition result display field 50. Preferably, the second mode is ended in response to the lapse of a certain period of time or in response to user input. The user input is preferably a simple operation such as pressing of the foot pedal or clicking of the mouse. Alternatively, means such as a switching button for switching to the first mode or the third mode may be included to correct a supplement to the recognition result or a detailed name of the recognition result or address apparent recognition errors.

Whether the reliability level is higher than the predetermined range is determined on the basis of a preset threshold value for the upper limit of the predetermined range. This threshold value is set as a first threshold value, and the second mode is set when the reliability level is greater than or equal to the first threshold value. Since the second mode is an acceptance mode in which a recognition result is confirmed without acceptance of a correction, it is preferable that the first threshold value be 90% or more.

As illustrated in FIG. 8, when the reliability level is lower than the predetermined range, the acceptance mode is switched to the third mode in which any input from the user is accepted. The display 14 displays the medical image 40 subjected to the recognition processing, an optional input acceptance field 51, a correction candidate display field 52, and a “Confirm” button 53. The optional input acceptance field 51 accepts input of any text from the user via the user interface 15. The correction candidate display field 52 displays a recognition result acquired by the recognition processing as reference information for the user to perform any input. Since the reliability level is low, unlike the first mode, selection of a correction candidate obtained as a recognition result is not accepted. The “Confirm” button 53 is a button for confirming the recognition result and ending the third mode after the user observes the medical image 40 and inputs the name of the specific photographic subject to the optional input acceptance field 51. The user operates the mouse or the like to select the “Confirm” button 53.

Whether the reliability level is lower than the predetermined range is determined on the basis of a preset threshold value for the lower limit of the predetermined range. This threshold value is a second threshold value, and the third mode is set when the reliability level is less than the second threshold value. Since the third mode is an acceptance mode in which a recognition result is confirmed by any input from the user when the recognition result is difficult to automatically confirm, the second threshold value is preferably less than 50%. Even when the reliability level is less than the first threshold value and greater than or equal to the second threshold value, the acceptance mode may be switched to the third mode if no other option is recognized in the recognition processing, for example, in the case of “tumor: 60%”.

The selection or determination of an item in the first to third modes is transmitted to the recognition result correction unit 34 or the recognition result confirmation unit 35 via the user interface 15. A user operation input using a mouse or a keyboard is available. Alternatively, the user operation may be input using any other means. For example, the foot switch may be used to select an option in the first mode, a gesture operation may be used to switch an acceptance screen in the second mode, and any text may be input in the third mode using audio input.

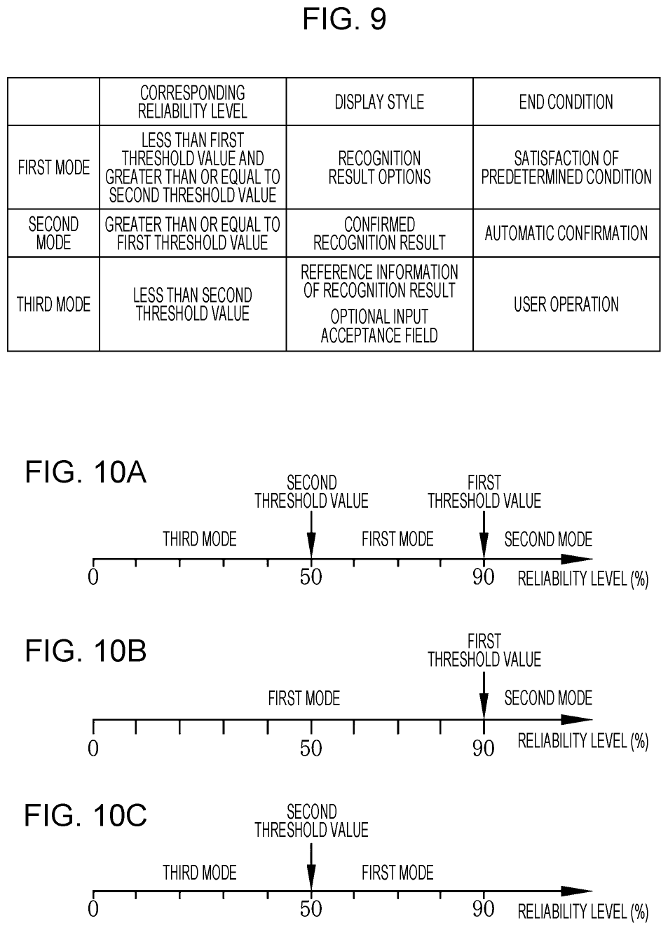

As illustrated in FIG. 9, each acceptance mode differs in terms of the value of the reliability level, the display style, and the procedure for confirming a recognition result and ending the acceptance mode. In the first mode in the case where the reliability level is less than the first threshold value and greater than or equal to the second threshold value, the user selects a recognition result from among a plurality of recognition result options. In the second mode in the case where the reliability level is greater than or equal to the first threshold value, no user operation on recognition results is accepted. In the third mode in the case where the reliability level is less than the second threshold value, reference information for recognition results acquired by the recognition processing is displayed to accept input of a recognition result in any text from the user.

The type of the specific photographic subject to be recognized in the recognition result is at least one of a lesion, a treatment tool, or a site to be observed. Before the recognition processing is performed, the type of the specific photographic subject to be detected in the recognition processing may be set in advance. Examples of the type include lesion detection for detecting only a lesion, treatment-tool detection for detecting only a treatment tool, and scene determination for determining a scene in which the medical image 40 of a site, an organ, or the like appears.

For each type of specific photographic subject to be recognized, at least one of the first threshold value for the reliability level or the second threshold value, which is lower than the first threshold value, may be set. For example, a small number of items are displayed as options for a treatment tool, and options can be determined by narrowing down the options by using endoscopic examination information. Thus, for the treatment tool, it is unlikely to be difficult to determine a recognition result from the options even when the second threshold value is set to a low value. However, even when the second threshold value is set individually for each type of specific photographic subject to be recognized, the second threshold value is lower than the first threshold value.

As illustrated in FIGS. 10A to 10C, in a modification of the present embodiment, instead of control of the three stages of acceptance modes, control of two stages of acceptance modes using only the first threshold value or the second threshold value may be performed. FIG. 10A illustrates control of three stages of acceptance modes using the first threshold value and the second threshold value described above. FIG. 10B illustrates control for accepting a correction to a recognition result by using only the first threshold value in either the first mode in which the reliability level is less than the first threshold value or the second mode in which the reliability level is greater than or equal to the first threshold value. FIG. 10C illustrates control for accepting a correction to a recognition result by using only the second threshold value in either the first mode in which the reliability level is greater than or equal to the second threshold value or the third mode in which the reliability level is less than the second threshold value.

In the case where only the first threshold value is used as in FIG. 10B, the control is performed without using the third mode, and a recognition result is automatically confirmed or confirmed when a predetermined condition such as the lapse of a certain period of time is satisfied. This configuration can reduce the number of operations performed by the user and enables a large number of recognition results to be confirmed in a short period of time. This control is preferably used for a recognition target such as a treatment tool that is classified by the recognition processing in a small number.

In the case where only the second threshold value is used as in FIG. 10C, the control is performed without using the second mode, and a recognition result is confirmed in response to selection of an option or in response to input of any text from the user. This configuration makes it possible to confirm the recognition result through input from the user without omitting observation of an image subjected to the recognition processing and omitting user input. This control is preferably used for a recognition target that is likely to be erroneously recognized, such as a rare lesion.

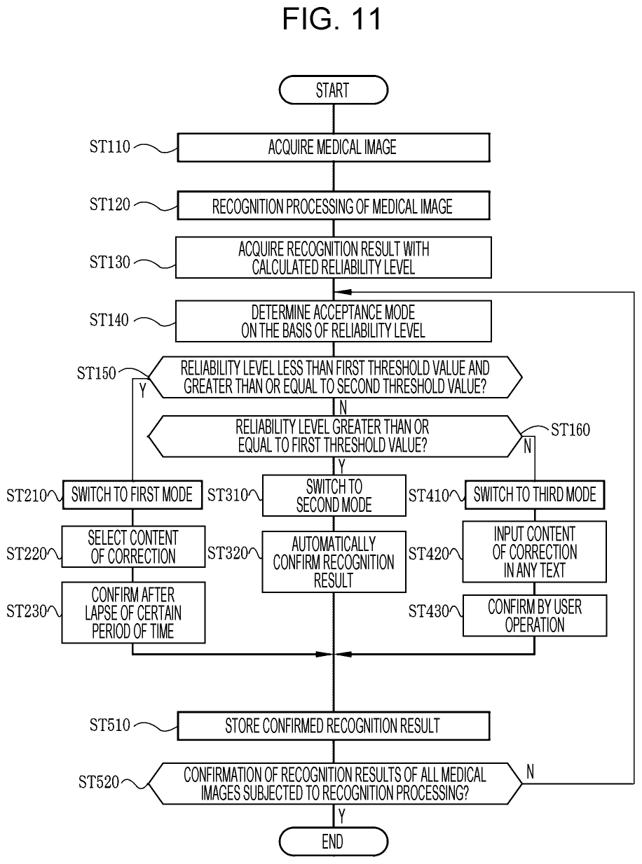

The flow of a series of operations for controlling a correction to a recognition result according to the present embodiment will be described with reference to a flowchart illustrated in FIG. 11. The medical image processing apparatus 11 acquires the medical image 40 captured through a medical examination from the database 12 or the endoscope system 13 (step ST110). The medical image processing apparatus 11 executes recognition processing to recognize a specific photographic subject included in the acquired medical image 40 (step ST120). The medical image processing apparatus 11 acquires a recognition result for the medical image 40 by the recognition processing together with a calculated reliability level (step ST130). The medical image processing apparatus 11 determines an acceptance mode for controlling acceptance of a correction to the recognition result on the basis of the reliability level of the medical image 40 (step ST140).

The medical image processing apparatus 11 determines whether the reliability level falls within a predetermined range, that is, less than the first threshold value and greater than or equal to the second threshold value (step ST150). If the reliability level is less than the first threshold value and greater than or equal to the second threshold value (Y in step ST150), the acceptance mode is switched to the first mode (step ST210). In the first mode, options that are candidates for recognition results are displayed on the screen, and the user observes the medical image 40 and determines a recognition result from any of the options (step ST220). In the first mode, the recognition result is confirmed when a predetermined condition such as the lapse of a certain period of time is satisfied (Step ST230). If the user does not select a recognition result, the item having the highest reliability level among the plurality of recognition results is determined to be the recognition result.

If the reliability level has a value outside the predetermined range (N in step ST150), the medical image processing apparatus 11 determines whether the reliability level has a value larger than or smaller than the predetermined range (step ST160). If the reliability level has a value larger than the predetermined range, that is, greater than or equal to the first threshold value (Y in step ST160), the acceptance mode is switched to the second mode (step ST310). In the second mode, the recognition result is automatically confirmed without acceptance of a correction to the recognition result (step ST320).

If the reliability level has a value smaller than the predetermined range, that is, less than the second threshold value (N in step ST160), the acceptance mode is switched to the third mode (step ST410). In the third mode, a correction to the recognition result is accepted by input of any text from the user (step ST420). In the third mode, in response to the user performing an operation such as pressing a confirmation button after inputting a recognition result in any text, the recognition result is confirmed (step ST430).

After the recognition result is confirmed, the medical image 40 associated with information on the confirmed recognition result is stored in the database 12 or the storage memory 23 (step ST510). If the recognition results of all the medical images 40 subjected to the recognition processing are not confirmed (N in step ST520), the medical image processing apparatus 11 determines an acceptance mode for controlling acceptance of a correction to the recognition result on the basis of the reliability level of the medical image 40, and continues the process of confirming the recognition result (step ST140). If the recognition results of all the medical images 40 subjected to the recognition processing are confirmed (Y in step ST520), the medical image processing ends.

### Second Embodiment

The first embodiment described above is an embodiment in which the reliability level calculated for each of the medical images 40 by the recognition processing is used to control acceptance of a correction to the recognition result. In contrast, an embodiment will be described in which, in response to acquisition of a medical image group 41 constituted by time-series medical images 40, a recognition result of the medical image group 41 is used to control acceptance of a correction to the recognition result. A description of content common to that of the embodiment described above will be omitted.

In recognition processing to be performed on a specific photographic subject such as a treatment tool from the time-series medical images 40, the same treatment tool is likely to be recognized in a certain unit time. For this reason, a change of recognition results of the treatment tool across the time-series medical images 40 can be determined to be erroneous recognition caused by a blur occurring in the recognition results. In addition to the recognition processing for the individual medical images 40 by machine learning, the use of the recognition result obtained for the entire time-series medical images 40, that is, the recognition result obtained for the medical image group 41, makes it possible to prevent erroneous recognition with high accuracy.

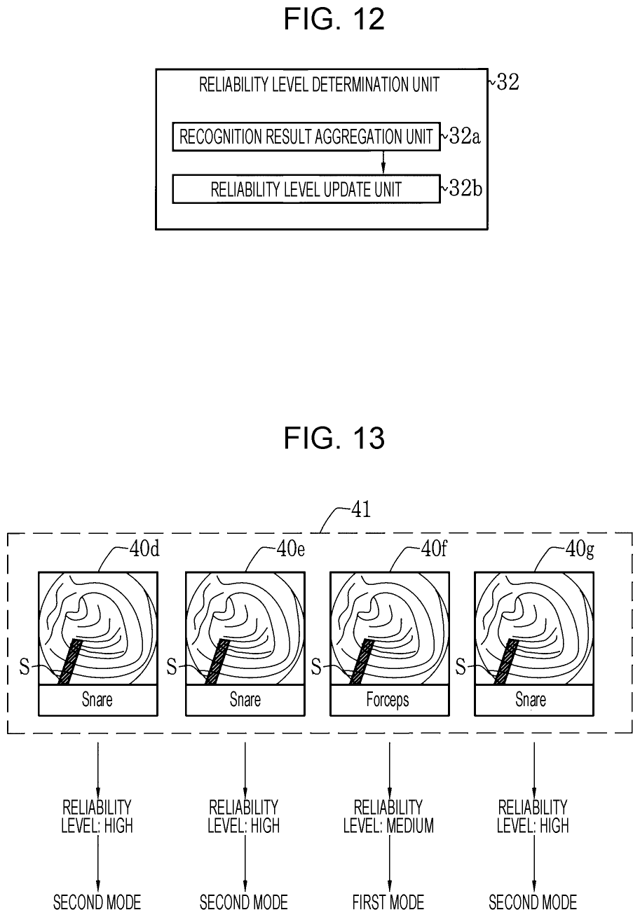

As illustrated in FIG. 12, to perform the recognition processing for the medical image group 41, the reliability level determination unit 32 implements the functions of a recognition result aggregation unit 32a and a reliability level update unit 32b. The recognition result aggregation unit 32a performs the recognition processing for the medical image group 41 by aggregating the recognition results of the constituent medical images 40. The recognition result of the medical image group 41 is determined by using the number of times a specific photographic subject having the same recognition result is recognized for each of the constituent medical images 40. The reliability level update unit 32b determines the reliability levels of the respective medical images 40, which are associated in time series on the basis of the recognition result of the medical image group 41, by updating the reliability levels from values calculated in the recognition processing. Acceptance of a correction to the recognition result is controlled.

The control of the acceptance mode using the recognition result of the medical image group 41 will be described. The medical image group 41 including the time-series medical images 40 is acquired from the database 12 or the endoscope system 13. In the recognition processing for the medical image group 41, the recognition processing is performed for each of the medical images 40, and a reliability level is calculated on the basis of the number of recognitions of a specific photographic subject with respect to the number of medical images 40 constituting the medical image group 41. The calculated reliability level is used to control acceptance of a correction to the recognition result for each of the medical images 40. The same recognition result accounting for the majority of the number of recognitions can be determined to be the recognition result of the medical image group 41. Alternatively, the number of times the same recognition result is consecutively obtained, rather than the same recognition result accounting for the majority, may be used.

In the control of the acceptance mode, the reliability level is updated based on whether the recognition results of the medical image group 41 and the medical image 40 match, and the acceptance mode is determined based on the updated reliability level. Alternatively, the acceptance mode may be determined based on whether the recognition results of the medical image group 41 and the medical image 40 match, without updating of the reliability level.

As illustrated in FIG. 13, the recognition processing for a medical image group 41 constituted by four time-series images will be described as an example. In a case where medical images 40d, 40e, and 40g of the “snare” and a medical image 40f of the “forceps” are acquired by the recognition processing for treatment-tool detection, three recognition results out of the four recognition results in the medical image group 41 are “snare”, and the recognition result of the medical image group 41 is “snare”. The medical images 40d, 40e, and 40g for which the same recognition result as that of the medical image group 41 is obtained have reliability levels that are increased to be greater than or equal to the first threshold value, and the acceptance mode is set to the second mode. The medical image 40f for which a recognition result different from that of the medical image group 41 is obtained has a reliability level that is decreased to be less than the first threshold value and greater than or equal to the second threshold value, and the acceptance mode is set to the first mode.

The recognition result aggregation unit 32a acquires a recognition result of the medical image group 41 including the time-series medical images 40 by using the same recognition result accounting for a certain proportion or more of the number of medical images 40, that is, the same recognition result that is obtained a certain number of times or more per unit time or is consecutively obtained a certain number of times or more. For example, in a case where the medical image group 41 is captured at a frame rate of 60 fps and the unit time is 1 second, among the recognition results obtained as a result of the recognition processing for 60 medical images 40 constituting the medical image group 41, the same recognition result obtained half the number of times (60) the recognition processing is performed, that is, 30 times or more, or the same recognition result consecutively obtained 20 times or more is used.

The reliability level update unit 32b can update the reliability level of each of the time-series medical images 40 from the recognition result of the medical image group 41. The reliability level of the recognition result of the medical image 40, which is the same as the recognition result of the medical image group 41, is updated to have a higher value. For example, the recognition processing of the treatment tool is performed, and “forceps” is acquired as the recognition result of the medical image group 41. In this case, the reliability level of “forceps” in each of the medical images 40 constituting the medical image group 41 is updated to have a larger value. The values are updated such that the larger the number of recognitions per unit time or the number of consecutive recognitions with respect to the number of medical images 40 constituting the medical image group 41, the larger the updated value.

Further, the reliability level of each of the time-series medical images 40 can be updated such that the reliability level of the medical image 40 for which a different recognition result from the recognition result of the medical image group 41 is obtained is updated to have a lower value. The values are updated such that the smaller the number of recognitions per unit time or the number of consecutive recognitions with respect to the number of medical images 40 constituting the medical image group 41, the smaller the updated value.

As a result of the update of the reliability level by the reliability level update unit 32b, the reliability level of the recognition result of each of the medical images 40 constituting the medical image group 41 is higher than that before the update when the recognition result is the same as the recognition result of the medical image group 41, and is lower than that before the update when the recognition result is different from the recognition result of the medical image group 41. This enables more accurate control of the acceptance mode. In addition, the recognition result may change due to a variation in the reliability level.

### Third Embodiment

The first embodiment and the second embodiment described above are embodiments in which recognition processing of one specific photographic subject is performed for each of the medical images 40 to control switching of the acceptance mode. The present embodiment provides a description of an embodiment in which a plurality of specific photographic subjects are recognized for one medical image 40. A description of content common to that of the embodiments described above will be omitted.

In recognition processing for recognizing a specific lesion type for which conditions for recognition are limited, site information is acquired in addition to the detection of the specific lesion type in the same medical image 40, and a combination thereof can be used to perform accurate reliability level calculation and recognition result acquisition for both specific photographic subjects.

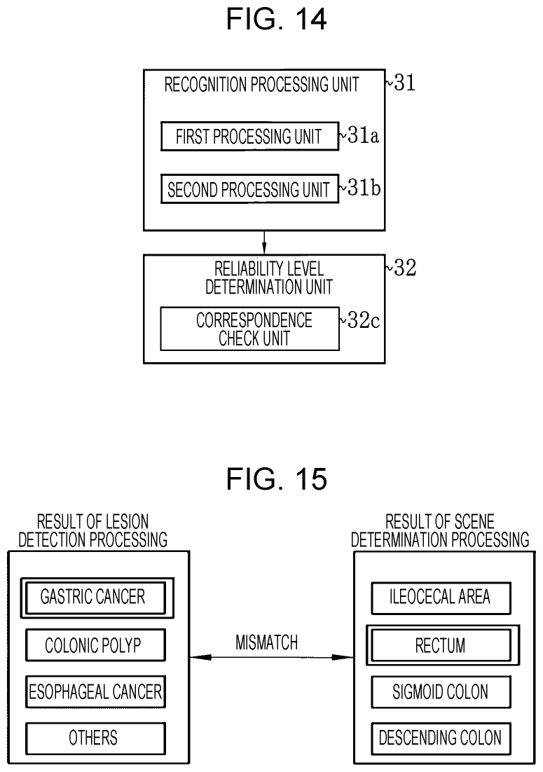

As illustrated in FIG. 14, to detect a plurality of specific photographic subjects from the medical image 40, the recognition processing unit 31 implements the functions of a first processing unit 31a and a second processing unit 31b, and the reliability level determination unit 32 implements the function of a correspondence check unit 32c. The reliability level of the medical image 40 for which the reliability levels of the plurality of specific photographic subjects are calculated is represented for each of the acquired recognition results.

The first processing unit 31a performs first recognition processing on the medical image 40 to recognize a specific photographic subject in a way similar to that of the recognition processing unit 31 in the embodiments described above and acquires a first recognition result. The second processing unit 31b executes second recognition processing on the medical image 40 to recognize a specific photographic subject and acquires a second recognition result. The second recognition processing recognizes a specific photographic subject of a different type from the type of the specific photographic subject recognized in the first recognition processing. The medical image 40 for which the first recognition result and the second recognition result are acquired is transmitted to the reliability level determination unit 32.

The reliability level determination unit 32 determines a first reliability level indicating the possibility of erroneous recognition of the first recognition result of the medical image 40 and a second reliability level indicating the possibility of erroneous recognition of the second recognition result. The correspondence check unit 32c stores in advance the correspondence between different types of specific photographic subjects, such as a combination of lesion information and site information. The correspondence check unit 32c checks whether the correspondence between the first recognition result and the second recognition result matches content stored in advance. In addition to the method for calculating a reliability level in the first embodiment, the first reliability level and the second reliability level are calculated on the basis of a matching result.

When the recognition results of the medical image 40 subjected to the two types of recognition processing are “tumor: 65%; bleeding: 20%; and gastritis: 15%” for the lesion and “pylorus: 40%; vestibule: 20%; cardia: 15%; and gastric body: 15%” for the site or organ, the medical image 40 is handled as “the reliability level for the lesion is 65%” and “the reliability level for the site is 40%”.

The switching of the acceptance mode is performed for each execution of recognition processing, and acceptance of a correction to each recognition result is controlled in a stepwise manner. For example, the same medical image 40 is subjected to acceptance of a correction in response to switching to an acceptance mode for a lesion and then acceptance of a correction in response to switching to an acceptance mode for a site. The order of corrections to be made to a plurality of recognition results for the same medical image 40 may be determined by the reliability level or may be determined by a user operation.

When the correspondence matches the stored content, the reliability levels are equal to or greater than those obtained without using the matching result, and when the correspondence does not match the stored content, the reliability levels are less than those obtained without using the matching result. When the correspondence does not match the stored content, the reliability levels are less than the first threshold value at most, that is, the value of the first mode or the third mode for accepting a correction, and a correction is performed. Alternatively, when the correspondence does not match the stored content, at least either result is the result of erroneous recognition. Thus, the reliability levels are set to 50%.

The check of the correspondence for a site in which a specific lesion type is likely to be detected will be described. The recognition processing performed by the first processing unit 31a executes detection processing for detecting a lesion to acquire a lesion detection result. The second recognition processing performed by the second processing unit 31b performs scene determination processing for determining a site or an organ to acquire a scene determination result. The correspondence check unit 32c stores in advance information on a combination of sites that can be detected for a specific lesion type, and checks whether the correspondence between the lesion detection result and the scene determination result, which are acquired, matches the stored information on the combination. The matching result indicating whether the correspondence matches the stored information is used to determine the first reliability level of the lesion detection result and the second reliability level of the scene determination result.

As illustrated in FIG. 15, if the correspondence between the lesion detection result and the scene determination result does not match the stored information, specifically, if the correspondence is such that the lesion recognition result is “gastric cancer” and the scene determination result is “rectum”, a mismatch is determined. Both the reliability level between which a mismatch is determined to be found, that is, the first reliability level of “gastric cancer” and the second reliability level of “rectum”, are updated to values lower than those before the correspondence is checked, and the acceptance mode control unit 33 switches the acceptance mode to the first mode or the third mode.

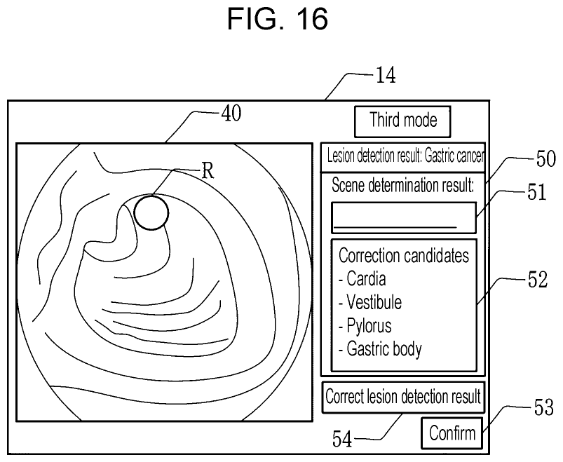

FIG. 16 illustrates a screen display of the display 14 for correcting the scene determination result when the first reliability level of “gastric cancer” is higher than the second reliability level of “rectum”, and the correction to the scene determination result is accepted in the third mode. The recognition result display field 50, which is displayed alongside the medical image 40, displays another recognition result in addition to the optional input acceptance field 51 and the correction candidate display field 52. Since none of “ileocecal area”, “sigmoid colon”, and “descending colon”, which are detected in addition to “rectum” in the scene determination processing, matches the correspondence with “gastric cancer”, the user inputs any text to the optional input acceptance field 51 to correct the scene determination result. The correction candidate display field 52 displays, as correction candidates, the respective sites of the stomach stored as combinations with “gastric cancer”, instead of the scene determination results. The input text can be confirmed by selecting the “Confirm” button 53. If the first reliability level of “gastric cancer” is lower than the second reliability level of “rectum”, it is preferable to switch to an acceptance mode for correcting the lesion detection result with the scene determination result set to be “rectum”. Whether to correct the correspondence by correcting the lesion detection result, correcting the scene determination result, or correcting both may be determined by a user operation such as selection of a correction target switching button 54.

Examples of the combination of a recognition result and a second recognition result include a combination of a lesion and a site or an organ, and a combination of a lesion and a treatment tool. In this case, the first processing unit 31a is set to perform lesion detection, the second processing unit 31b is set to perform treatment-tool detection processing, and the correspondence check unit 32c stores the types of treatment tools that can be used for a specific lesion. The first processing unit 31a and the second processing unit 31b may be set reversely.

In a modification of the third embodiment, recognition processing of a plurality of types of specific photographic subjects may be executed on the medical image 40 without implementation of the functions of the correspondence check unit 32c to acquire respective recognition results, reliability levels may be individually calculated, and the switching of the acceptance mode may be controlled. For example, the first processing unit 31a performs lesion detection processing as the first recognition processing, and the second processing unit 31b performs scene determination processing as the second recognition processing. Then, reliability levels are calculated for the respective types of recognition processing, and control is performed to accept a correction to the recognition results. That is, the acceptance mode for each of the recognition results that are not associated with each other is controlled for one medical image 40, and a correction is accepted.

Alternatively, in addition to the first processing unit 31a and the second processing unit 31b, the functions of a third processing unit (not illustrated) may be implemented to execute recognition processing of three types of specific photographic subjects in one medical image 40 and individually calculate the respective reliability levels of the three types of specific photographic subjects.

The present embodiment has been described using an example in which the database 12 to which the medical image processing apparatus 11 is connected is connected to the endoscope system 13 to process an endoscopic examination image acquired by the endoscope 13a. However, the present invention is not limited to this, and recognition processing may be performed on a medical image 40 acquired by any other medical examination apparatus or the like, such as an ultrasound imaging apparatus or a radiographic imaging apparatus to determine the presence or absence and name of a specific photographic subject.

In the embodiments described above, the hardware structures of the central control unit 20, the image acquisition unit 21, the output control unit 24, the input receiving unit 22, and processing units that execute various processes and are included in the recognition unit 30, such as the recognition processing unit 31, the reliability level determination unit 32, the acceptance mode control unit 33, the recognition result correction unit 34, and the recognition result confirmation unit 35, are various processors described below. The various processors include a CPU (Central Processing Unit), which is a general-purpose processor executing software (program) to function as various processing units, a programmable logic device (PLD) such as an FPGA (Field Programmable Gate Array), which is a processor whose circuit configuration is changeable after manufacturing, a dedicated electric circuit, which is a processor having a circuit configuration specifically designed to execute various types of processing, and so on.

A single processing unit may be configured as one of the various processors or as a combination of two or more processors of the same type or different types (for example, a plurality of FPGAs or a combination of a CPU and an FPGA). Alternatively, a plurality of processing units may be configured as a single processor. Examples of configuring a plurality of processing units as a single processor include, first, a form in which, as typified by a computer such as a client or a server, the single processor is configured as a combination of one or more CPUs and software and the processor functions as the plurality of processing units. The examples include, second, a form in which, as typified by a system on chip (SoC) or the like, a processor is used in which the functions of the entire system including the plurality of processing units are implemented as one IC (Integrated Circuit) chip. As described above, the various processing units are configured by using one or more of the various processors described above as a hardware structure.

More specifically, the hardware structure of these various processors is an electric circuit (circuitry) in which circuit elements such as semiconductor elements are combined. The hardware structure of a storage unit (memory) is a storage device such as an HDD (hard disc drive) or an SSD (solid state drive).

### REFERENCE SIGNS LIST

10 medical image processing system
11 medical image processing apparatus
12 database
13 endoscope system
13a endoscope
14 display
15 user interface
20 central control unit
21 image acquisition unit
22 input receiving unit
23 storage memory
24 output control unit
30 recognition unit
31 recognition processing unit
31a first processing unit
31b second processing unit
32 reliability level determination unit
32a recognition result aggregation unit
32b reliability level update unit
32c correspondence check unit
33 acceptance mode control unit
34 recognition result correction unit
35 recognition result confirmation unit
40 medical image
40a medical image
40b medical image
40c medical image
40d medical image
40e medical image
40f medical image
40g medical image
41 medical image group
50 recognition result display field
51 optional input acceptance field
52 correction candidate display field
53 “Confirm” button
54 correction target switching button
C cursor
R region of interest
S treatment tool

What is claimed is:

1. A medical image processing apparatus comprising:
one or more processors configured to:
acquire at least one medical image;
execute recognition processing on the medical image to recognize at least one specific photographic subject, the type of the specific photographic subject being at least one of a lesion, a treatment tool, or a site to be observed;
calculate a reliability level of a recognition result of the specific photographic subject;
determine, based on the reliability level, an acceptance mode for accepting a correction to the recognition result;
accept the correction to the recognition result in the determined acceptance mode; and
display the recognition result that is corrected.

2. The medical image processing apparatus according to claim 1, wherein
the acceptance mode includes a first mode for accepting a correction to the recognition result from a user and automatically confirming the correction to the recognition result in a case where a predetermined condition is satisfied.

3. The medical image processing apparatus according to claim 2, wherein the first mode
displays, as options, candidate corrections to the recognition result, and
allows the user to select a correction to the recognition result from the candidate corrections.

4. The medical image processing apparatus according to claim 2, wherein
the acceptance mode includes a second mode for confirming the recognition result without accepting a correction to the recognition result from the user.

5. The medical image processing apparatus according to claim 4, wherein the one or more processors are configured to:
determine the acceptance mode to be the first mode in a case where the reliability level is less than a first threshold value; and
determine the acceptance mode to be the second mode in a case where the reliability level is greater than or equal to the first threshold value.

6. The medical image processing apparatus according to claim 2, wherein
the acceptance mode includes a third mode for accepting a correction to the recognition result from the user and manually confirming the recognition result.

7. The medical image processing apparatus according to claim 6, wherein the one or more processors are configured to:
determine the acceptance mode to be the first mode in a case where the reliability level is greater than or equal to a second threshold value; and
determine the acceptance mode to be the third mode in a case where the reliability level is less than the second threshold value.

8. The medical image processing apparatus according to claim 1, wherein the one or more processors are configured to:
acquire time-series images that are the medical images in time series;
perform the recognition processing for each of the medical images constituting the time-series images; and
calculate the reliability level, based on a number of recognitions of the specific photographic subject with respect to a number of the medical images constituting the time-series images.

9. The medical image processing apparatus according to claim 8, wherein the one or more processors are configured to:
calculate the reliability level, based on a number of recognitions of a photographic subject different from the specific photographic subject with respect to the number of medical images constituting the time-series images.

10. The medical image processing apparatus according to claim 1, wherein the one or more processors are configured to:
execute, in the recognition processing, first recognition processing for recognizing the specific photographic subject and second recognition processing for recognizing a photographic subject different from the specific photographic subject;
acquire a first recognition result by the first recognition processing;
acquire a second recognition result by the second recognition processing;
check a correspondence between the first recognition result and the second recognition result; and
calculate a first reliability level of the first recognition result and a second reliability level of the second recognition result.

11. The medical image processing apparatus according to claim 1, wherein the one or more processors are configured to:
execute the recognition processing on the medical image for the plurality of mutually different specific photographic subjects;
acquire each of the recognition results; and
calculate the reliability level for each of the recognition results.

12. The medical image processing apparatus according to claim 1, wherein the one or more processors are configured to:
set at least one of a first threshold value of the reliability level or a second threshold value lower than the first threshold value for each type of the specific photographic subject; and
determine the acceptance mode, based on the reliability level for at least one of the first threshold value or the second threshold value.

13. The medical image processing apparatus according to claim 1, wherein the one or more processors are configured to:
control acceptance of the correction and a display style of a screen in a stepwise manner, based on the reliability level.

14. The medical image processing apparatus according to claim 1, wherein the one or more processors are configured to:
accept a button operation, a foot pedal operation, and a voice input for the correction.

15. A method for operating a medical image processing apparatus, the method comprising:
a step of acquiring at least one medical image;
a step of executing recognition processing on the medical image to recognize at least one specific photographic subject, the type of the specific photographic subject being at least one of a lesion, a treatment tool, or a site to be observed;
a step of calculating a reliability level of a recognition result of the specific photographic subject;
a step of determining, based on the reliability level, an acceptance mode for accepting a correction to the recognition result;
a step of accepting the correction to the recognition result in the determined acceptance mode; and
a step of displaying the recognition result that is corrected.
